(19) United States
(12) Patent Application Publication
(10) Pub. No.: US 2018/0114598 A1
(43) Pub. Date: Apr. 26, 2018

(54) METHOD AND APPARATUS FOR PROGRAMMING A MEDICATION DELIVERY DEVICE

(71) Applicant: Codonics, Inc., Middleburg Heights, OH (US)

(72) Inventors: Michael KOLBERG, Hinckley, OH (US); Gary KEEFE, Brecksville, OH (US); Peter BOTTEN, Lakewood, OH (US)

(73) Assignee: Codonics, Inc., Middleburg Heights, OH (US)

(21) Appl. No.: 15/569,904

(22) PCT Filed: Apr. 28, 2016
(86) PCT No.: PCT/US16/29766

§ 371 Date: Oct. 27, 2017

Related U.S. Application Data
(60) Provisional application No. 62/153,661, filed on Apr. 28, 2015.

(51) Int. Cl. G16H 20/17; G16H 10/60

(52) U.S. Cl. CPC G16H 20/17; G16H 10/60

(57) ABSTRACT

Provided is a method and system for programming a drug delivery device. The method includes using a portable computer terminal to read a device code encoding information specific to the drug delivery device, a patient code identifying a specific patient that is to receive the drug administered by the drug delivery device, and a drug code accompanying a container storing the drug to be administered to the specific patient by the drug delivery device. Once input is received from a user indicating that operation of the drug delivery device to deliver the drug to the specific patient is to begin, the portable computer terminal initiates creation of an electronic record thereon linking the information specific to the drug delivery device, the information indicative of the identity of the specific patient, and the information indicative of the drug delivery parameter.

### BACKGROUND OF THE INVENTION

### 1. Field of the Invention

This application relates generally to a method and apparatus for programming a medication delivery device.

### 2. Description of Related Art

The process of administering medications to patients in healthcare institutions such as hospitals involves a number of steps that are typically recorded to the patient's medical record. These steps can be spread across multiple departments in the healthcare institution and commonly include the patient's physician that orders the medication, the pharmacist that prepares the medication and the nurse that administers the medication to the patient. In recent years, electronic medical record systems (“EMR”) have been increasingly used for recording information about events such as administering medications to patients. In some cases, the EMR can even transmit information about the medication to be administered over a private local area network (“LAN”) implemented at the healthcare institution to devices like infusion pumps that deliver medication to the patient to assist nurses in programming the device. This can be useful to reduce user errors caused by programming error on multiple devices with different user interfaces being used within a healthcare institution.

Most of the medication administration workflow within a healthcare institution involves transmitting information about the medication over the LAN to be stored as part of the patient's medical record as each step of the process is performed. This has the advantage of keeping the patient's medical record current, but can also lead to problems when medical records are maintained electronically and technical issues prevent clinicians from accessing those records when a critical medication must be administered. For example, interruptions in LAN communications (e.g., connection to a server is lost, a router/hub is not functioning properly, enhanced security required of LAN communications at healthcare institutions interferes with network communications, etc.) may prevent electronic communication between the EMR and the infusion pump being utilized.

### BRIEF SUMMARY OF THE INVENTION

According to one aspect, the subject application involves a method of programming a drug delivery device. The method includes using a portable computer terminal to read a device code associated with the drug delivery device. The device code is in a computer-readable format, which is not readily interpreted by the naked eye of a human observer without the assistance of a computer reader, which encodes information specific to the drug delivery device that is interpreted by the portable computer terminal. The portable computer terminal is also used to read a patient code in a computer-readable format encoding information indicative of an identity of a specific patient that is to receive the drug administered by the drug delivery device that is interpreted by the portable computer terminal. The portable computer terminal is also used to read a drug code accompanying a container storing the drug to be administered to the specific patient by the drug delivery device. Again, the drug code is in a computer-readable format encoding information indicative of a drug delivery parameter for administering the drug to the specific patient with the drug delivery device that is interpreted by the portable computer terminal. Input is received from a user indicating that operation of the drug delivery device to deliver the drug to the specific patient is to begin. In response to receiving this input, the portable computer terminal initiates creation of an electronic record thereon linking the information specific to the drug delivery device, the information indicative of the identity of the specific patient, and the information indicative of the drug delivery parameter.

The above summary presents a simplified summary in order to provide a basic understanding of some aspects of the systems and/or methods discussed herein. This summary is not an extensive overview of the systems and/or methods discussed herein. It is not intended to identify key/critical elements or to delineate the scope of such systems and/or methods. Its sole purpose is to present some concepts in a simplified form as a prelude to the more detailed description that is presented later.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 shows an illustrative embodiment of a gravity-fed, intravenous bag fluid infusion device that administers predetermined quantities of a drug to a patient;

FIG. 2 shows an illustrative embodiment of a pump fluid infusion device that actively administers predetermined quantities of a drug to a patient; and

FIG. 3 shows an illustrative embodiment of a local area network (“LAN”) established at a healthcare institution for programming a plurality of different fluid infusion devices to administer predetermined quantities of drugs to patients and monitor and record information pertaining to such administration.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

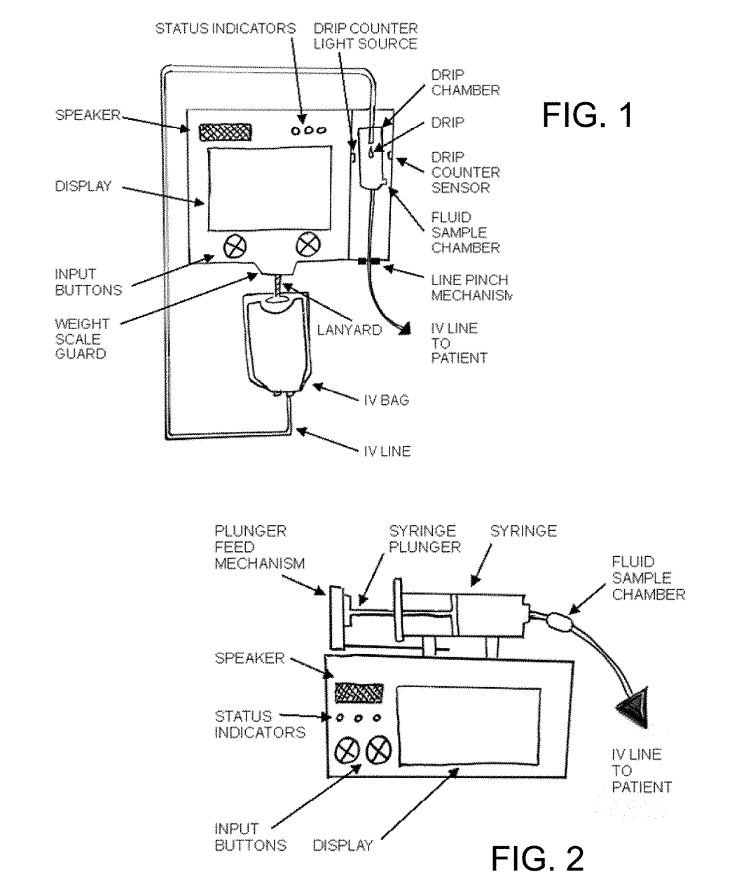

FIG. 1 schematically depicts an illustrative embodiment of a type of infusion pump referred to as an IV bag fluid infusion device. As shown, the IV Bag containing the drug to be administered is hung from a hook, referred to in FIG. 1 as a lanyard, which is optionally coupled to a scale or other weight sensing device. The drug flows through the IV line under the force of gravity to a drip chamber equipped with a drop count sensor and drip counter light source to sense each drop emitted from the IV line, allowing the drops to be counted by a processing unit provided to the infusion device or “pump”. The rate of delivery can be adjusted by the pump by controlling the degree to which the IV line to the patient is constricted or “pinched” by the pump.

FIG. 2 schematically depicts another illustrative embodiment of a type of infusion pump referred to as a pump fluid infusion device, or syringe pump. According to the present embodiment, infusion device receives a syringe storing the drug to be delivered and a plunger feed mechanism that inserts the plunger, and accordingly, administers the drug at a controlled rate through the IV line to the patient according to the parameters programmed into the infusion device. Although two types of infusion devices are shown in FIGS. 1 and 2 and reference herein for the sake of brevity and clarity, the present disclosure can encompass any other device for administering a fluid to a patient in a controlled manner. Thus, the infusion pumps will be generically referred to herein as Pumps.

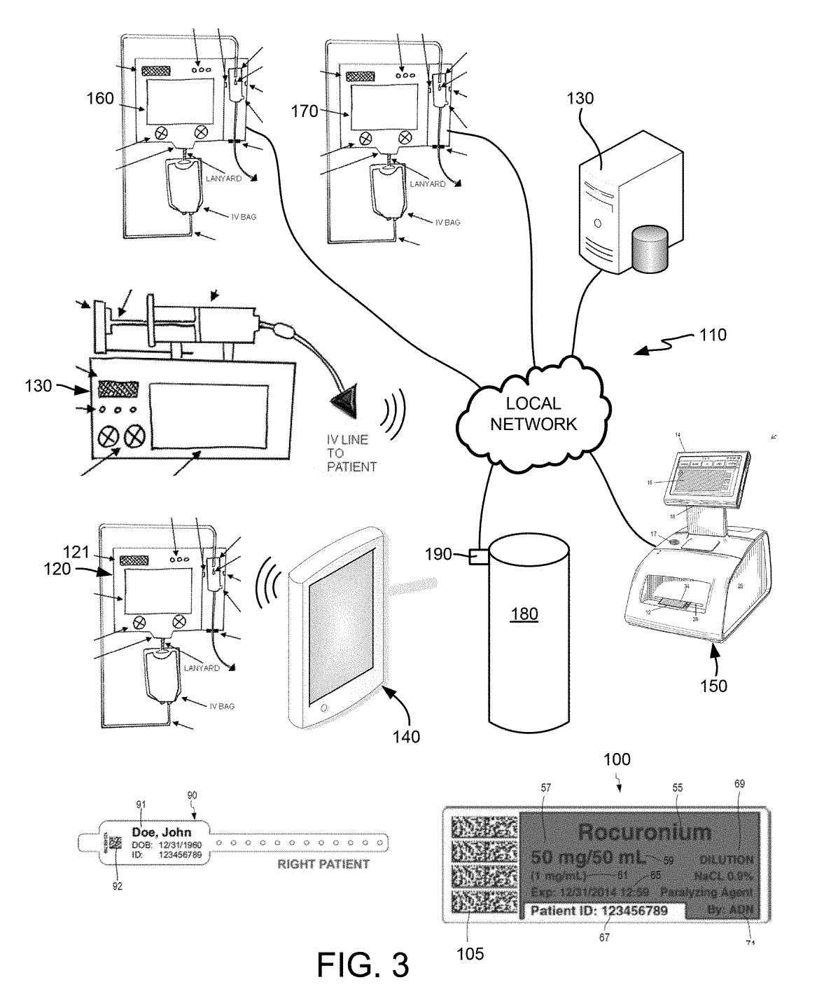

The Pumps can optionally lack network communication abilities and hardware (e.g., devoid of a communication portion such as a RJ45 Ethernet port, BlueTooth or IEEE 802.11 antenna, etc.) altogether, and/or optionally be equipped with varying network communication abilities and hardware to allow communications with other devices operatively connected to a LAN 110, as shown in FIG. 3. For example, Pump 115 in FIG. 3 lacks the hardware and/or software required to facilitate communications of programming parameters governing the administration of a drug utilizing the Pump 115 to the Pump 115 from another network resource over the LAN 110. To promote network security, the LAN 110 can optionally be site specific, and optionally lack a connection to a publicly-accessible wide area network (“WAN”) such as the Internet, or employ security features to interfere with the ability of unauthorized parties to access the LAN 110.

The syringe, IV bag or other container from which the drug is to be administered can optionally be provided with a label 100, shown in FIG. 3, which includes a computer-readable code 105 (e.g., barcode, RFID tag, etc.) encoding information pertaining to the administration of the drug utilizing a Pump in a data format that is readable by a compatible computer-implemented reader. The code 105 can optionally encode at least a portion of human-readable content (e.g., alpha and/or numeric characters discernable via the human eye and understandable without translation from a machine readable format) appearing on the label 100, and/or at least a portion of, and optionally all of the parameters governing the administration of the drug to be administered via the Pump. An example of a labeling system 150 (FIG. 3) for preparing such a label 100 is described in U.S. Pat. No. 8,639,525 to Levine et al., which is incorporated in its entirety herein by reference. Such labels 100 are adhesive-backed labels printed using a computer printer to include a barcode. The labels 100 of this embodiment can be considered suitable for a one-time use, and disposed of along with the empty container following completion of the infusion.

According to other embodiments, the code 105 can be embodied in a readable and writable form, such as a RFID tag for example. The RFID tag can optionally be coupled to the IV bag, syringe or other container in a removable, reusable fashion to allow the RFID tag to be formatted and subsequently reused to label a different container once a drug in a first container labeled with the RFID tag has been administered. For example, the RFID tag can be provided to a hangar coupled to the IV bag. Such hangars can be releasable, meaning they can be coupled to a first IV bag, for example, and subsequently removed from the IV bag and coupled to another IV bag containing a drug for a subsequent, different infusion.

Regardless of the format of the code 105 provided to the label 100, the Pumps can optionally be equipped with a compatible code reader (e.g., barcode scanner, RFID antenna and reader component, etc.) to interrogate and read a computer-readable code such as the code 105 provided to a label coupled to the IV bag, syringe or other container. The code 105 and label 100 can optionally be provided at a known location on or adjacent to the IV bag, syringe or other container so the code reader provided to the Pump can read the code 105 when the container is positioned on the Pump for administration of the drug. In other words, no special arrangement of the container other than the arrangement required for administering the drug using the Pump is required. Thus, parameters governing infusion of the drug via the Pump encoded by the code 105 can be delivered and programmed into the Pump simply by hanging the IV bag, inserting the syringe, or otherwise coupling any other container to the Pump for an infusion.

For embodiments where code 105 is positioned for reading and/optionally writing while the drug is being administered, information concerning certain triggering events that take place during administration of the drug can optionally be written to writable embodiments of the code 105 (e.g., RFID tag) before, during and/or after administration of the drug. For example, if drug delivery is interrupted for whatever reason, the RFID reader provided to the Pump can write information detailing the interruption to the code 105. Such information can include at least one of: the point during the administration the interruption occurred (e.g., progress of the infusion), the quantity of the drug delivered at the time of the interruption, the duration of the interruption, whether administration of the drug was resumed, etc. The information written to the code 105 is not limited to interruptions, but can include any type of noteworthy event that could affect the provision of healthcare to the patient. For instance, the code reader provided to the Pump can optionally write information identifying the specific Pump used to administer a drug, the identity of the clinician who prepared the medication, the identify of the clinician who programmed the Pump, the identity of the clinician who ordered and is ultimately responsible for the infusion (e.g., the patient's primary physician), the identity of the patient, etc. Further, a log/history detailing a plurality of events that occurred throughout the infusion can be written to the code 105 for subsequent analysis and documentation purposes as described below. Certain interruptions may occur during an infusion that may prevent the Pump from resuming the infusion. For instance, the Pump experiences a fatal malfunction. Under such circumstances, the information written to the code 105 can subsequently be read by, or otherwise entered into a different Pump that is functioning properly to resume the infusion that was interrupted.

FIG. 3 shows an illustrative embodiment of a LAN 110 established at a healthcare institution. The LAN 110 includes a plurality of different Pumps to administer predetermined quantities of drugs to patients and monitor and record information pertaining to such administration. The Pumps are said to be different in that they may be from different manufacturers, constitute different models (e.g., a state-of-the-art Pump and a legacy version of the Pump) from the same or different manufacturers, can include different hardware and/or capabilities, or can simply be programmed differently than other Pumps in the LAN 110. For instance, Pump 120 may be an outdated, legacy model that lacks the resources to communicate via the LAN 110. Although the Pump 120 may not be able to communicate with an EMR 125, for example, over the LAN 110, the Pump 120 can optionally be equipped with the hardware and software components to communicate directly with a local programmer module positioned within a predetermined range (e.g., a few feet), which can be thought of as a remote control for that Pump 120. Different Pumps such as Pump 120 and Syringe Pump 130, which constitute different models that can require programming with different operating parameters and may possibly even be manufactured by different manufacturers. Programming the Pumps 120, 130 can be accomplished using a portable terminal 140 such as a tablet computer (e.g., Apple iPad, Apple iPhone, Android tablet, etc.). The portable terminal 140 can optionally follow a workflow and/or optionally display a user interface having a common “look and feel” to program a plurality of different Pumps. Although the substantive content presented to a clinician programming the different Pumps may differ depending on the Pumps being programmed (e.g., request different types of information specific to the different pumps, etc.), the workflow for programming the Pumps and the appearance of the user interface will be familiar to the clinician, regardless of the type of Pump being programmed. The common workflow and/or look and feel will provide clinicians a degree of comfort, allowing them to effectively program different Pumps with a sense of familiarity.

The collection of information can begin with an electronic prescription or drug order submitted for a patient. A pharmacy may utilize a labeling system 150 to print a label 100 or otherwise prepare a label (e.g., write information to a RFID tag, etc.) to be coupled to the IV bag, syringe or other container from which the drug is to be administered to the patient. With the labeling system 150, the pharmacist can interrogate a computer-readable code associated with the prescription or drug order identifier using a compatible reader (e.g., barcode reader) provided to the labeling system 150, or receive the prescription and/or order information from a network-connected terminal such as the EMR 160 over the LAN 110. The information received by these means other than through manual entry into the labeling system 150 can include at least one of: patient identity (e.g., ID number, date of birth, name, etc.), drug name, drug delivery rate, duration of infusion, total drug volume, and total dose, etc. Receiving such information in a manner that does not require manual entry into the labeling system 150 helps to minimize the opportunities for human error.

At least a portion of the received information is written to the code 105, which is then coupled to the IV bag, syringe or other container. At the point of care, the clinician who is to initiate the infusion approaches the Pump 120 and scans or otherwise reads a computer-readable code 121 applied to or otherwise associated with the Pump 120 using the portable terminal 140. The portable terminal 140 can optionally include a hard drive or other storage medium locally storing information suitable to allow the portable terminal 140 to identify the Pump 120 based on the code 121. Thus, the portable terminal 140 does not necessarily have to rely in the availability or proper functioning of the LAN 110 to perform the steps described herein. According to alternate embodiments, the pump-identifying information can be retrieved from a storage location accessible via a WAN using a built-in telecommunication component (e.g., SIM card, cellular communication antenna) independently of the LAN 110.

In other embodiments, the portable terminal 140 can optionally include a hard drive or other storage medium locally storing information enabling the terminal to made decisions affecting the administration of the drug based on rules and data stored in the terminal. These can include for example, limits on the flow rate, type of drug and volume of drug or fluid delivered based on the drug, pump or patient information received by the terminal.

The clinician can then scan the code 105 provided to the label 100 coupled to the IV bag to identify the drug-specific parameters required to program the Pump 120 for this particular infusion. Again, the portable terminal 140 can reference a local formulary and/or other database locally stored on the storage medium provided to the portable terminal 140 to allow identification of the drug programming parameters for this particular infusion without the need to reference a remotely-located database over the LAN 110. According to alternate embodiments, the drug information can be retrieved from a storage location accessible via a WAN using the built-in telecommunication component of the portable terminal 140.

To confirm the identity of the patient receiving the drug, the portable terminal 140 can be used to read the computer-readable code 92 provided to a wristband 90 being worn by the patient. The portable terminal 140 can compare the patient's identity based on the code 92 to patient information encoded by the code 105 provided to the label 100 coupled to the IV bag. The portable terminal 140 can optionally display the patient and/or drug information for manual confirmation purposes to the clinician. Once all information has been determined to be in conformance, the clinician can instruct the portable terminal 140 to program the Pump 120 with the appropriate parameters for this particular infusion.

Since the Pump 120 lacks the ability to communicate over the LAN 110, the portable terminal 140 can optionally create an electronic record including the information used to program the Pump 120 and the patient information. The portable terminal 140 can optionally communicate with the EMR 130 over the LAN 110 or optionally be physically plugged into a communication hub operatively connected to the EMR 130 to transmit the contents of the electronic record to the EMR 130 once the Pump 120 has been programmed. Thus, the portable terminal 140 can convey information from a legacy Pump 120 that lacks network compatibility over the LAN 110 for record keeping, and optionally automate the programming of such a Pump 120.

However, the Pump 120 may also lack even the ability to communicate locally with the portable terminal 140, requiring the manual entry of the parameters (e.g., delivery rate, delivery time, etc.) governing the infusion. For such embodiments, the portable terminal 140 can still be utilized as described above, except for the step of programming the Pump 120. Instead, the portable terminal 140 will display the parameters and the programming instructions for manual entry. The portable terminal 140 can also optionally display an automated graphic simulating the drip rate that should be observed for this particular infusion.

According to alternate embodiments, Pumps 130, 160, 170 with LAN-communication ability can optionally be programmed as described above using the portable terminal 140. Additionally, such Pumps 130, 160, 170 can optionally also include a transceiver to transmit data in real-time over the LAN 110 and/or write data locally to the writable code 150 coupled to the drug container to store data concerning the infusion. Following completion of the infusion, the drug container can optionally be disposed of in a waste container 180 provided with a code reader 190 operatively connected to the LAN 110. When the drug container is introduced to the waste container 180, the reader 190 can read the information encoded by the code 105 to document the extent to which the infusion was completed. This information can be conveyed by the code reader 190 over the LAN 110 for storage in the EMR or other storage location in association with patient's records.

According to alternate embodiments, where the code 105 is not writable, the portable terminal 140 can optionally resume communications with the Pump 120 to obtain information that would have otherwise been written to the code 105 to obtain the information pertaining to the extent to which the infusion was completed. This information can optionally then be conveyed to the EMR 130 from the portable terminal 140 via the LAN 110 as described above.

Illustrative embodiments have been described, hereinabove. It will be apparent to those skilled in the art that the above devices and methods may incorporate changes and modifications without departing from the general scope of this invention. It is intended to include all such modifications and alterations within the scope of the present invention. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A method of programming a drug delivery device, the method comprising:
with a portable computer terminal, reading a device code associated with the drug delivery device, the device code being in a computer-readable format that encodes information specific to the drug delivery device that is interpreted by the portable computer terminal;
with the portable computer terminal, reading a patient code, the patient code being in a computer-readable format encoding information indicative of an identity of a specific patient that is to receive the drug administered by the drug delivery device that is interpreted by the portable computer terminal;
with the portable computer terminal, reading a drug code accompanying a container storing the drug to be administered to the specific patient by the drug delivery device, the drug code being in a computer-readable format encoding information indicative of a drug delivery parameter for administering the drug to the specific patient with the drug delivery device that is interpreted by the portable computer terminal;
receiving input from a user indicating that operation of the drug delivery device to deliver the drug to the specific patient is to begin; and
in response to said receiving input, initiating creation of an electronic record with the portable computer terminal linking the information specific to the drug delivery device, the information indicative of the identity of the specific patient, and the information indicative of the drug delivery parameter.

2. The method of claim 1 further comprising transmitting the electronic record to a computer system for documentation over a local area network.

3. The method of claim 2, wherein said transmitting the electronic record comprises:
storing the electronic record locally on the portable terminal at a point of care where the drug delivery device is located; and
subsequent to configuration of the drug delivery device, transmitting the electronic record locally stored to a remotely-located server that manages patient records.

4. The method of claim 1, wherein said reading the device code comprises interrogating a computer-readable code coupled to, or adjacent to an infusion pump that lacks an ability to communicate with a computer terminal over a communication network using a network communication protocol.

5. The method of claim 4 further comprising displaying, with the portable terminal, instructional information to be followed by a clinician to program the infusion pump with the drug delivery parameter obtained in response to reading the drug code.

6. The method of claim 5, wherein the instructional information is specific to a type of the drug delivery device, and different from other instructional information corresponding to a different drug delivery device.

7. The method of claim 6, wherein the instructional information and the other instructional information are shown using a common user interface.

8. The method of claim 1 further comprising transmitting the drug delivery parameter from the portable terminal to be received by the drug delivery device electronically.

9. The method of claim 8, wherein the drug delivery parameter is transmitted directly by the portable terminal to the drug delivery device via a communication channel.

10. The method of claim 8, wherein the drug delivery parameter is transmitted from the portable terminal to the drug delivery device over a local communication network.

11. The method of claim 1 further comprising, with the portable computer terminal, receiving and storing information indicative of an event involving the drug delivery device that occurred during administration of the drug to the patient.
